(19) United States
(12) Patent Application Publication
(10) Pub. No.: US 2018/0214506 A1
(43) Pub. Date: Aug. 2, 2018

(54) COMBINATION ADMINISTRATION OF GNRH ANTAGONISTS

(71) Applicant: ANTEV LIMITED, London (GB)

(72) Inventor: Finn LARSEN, Hawick (GB)

(21) Appl. No.: 15/884,108

(22) Filed: Jan. 30, 2018

Related U.S. Application Data
(60) Provisional application No. 62/452,096, filed on Jan. 30, 2017.

(51) Int. Cl. A61K 38/09

(52) U.S. Cl. CPC A61K 38/09

(57) ABSTRACT

Disclosed herein are methods of administering to a subject a GnRH antagonist with enhanced bioavailability. Also disclosed herein are methods of treating or preventing a disease or condition by administering a GnRH antagonist, and kits comprising the GnRH antagonist.

### CROSS REFERENCE

The present application claims the benefit of priority to Provisional Application No. 62/452,096, filed Jan. 30, 2017; which is incorporated herein by reference in its entirety.

### SUMMARY

Disclosed herein are methods comprising administering to a subject a therapeutically effective amount of a GnRH antagonist or a salt thereof, where the administering of the GnRH antagonist or salt thereof can produce a maximal plasma concentration (Cmax) of at least about 10 ng/mL and an area under the plasma concentration versus time curve from time 0 to time t (AUC₍₀₋ₜ₎) from about 100 ng*h/mL to about 8,000 ng*h/mL when administered at a total dose of from about 60 mg to about 480 mg, and where t can be at least about 28 days. In some instances, the administering of the GnRH antagonist or salt thereof can produce a Cmax of at least about 15 ng/mL and an AUC₍₀₋ₜ₎ from about 1,000 ng*h/mL to about 3,000 ng*h/mL when administered at a total dose of from about 120 mg to about 240 mg, and where t can be at least about 28 days. In some instances, the GnRH antagonist can be administered at a total dose of about 180 mg. In some instances, the administering can comprise an administering intramuscularly and an administering subcutaneously of the GnRH antagonist of salt thereof. In some instances, the administering intramuscularly and the administering subcutaneously can independently comprise administering a dose ranging from about 30 mg to about 240 mg of the GnRH antagonist or salt thereof. In some instances, the administering intramuscularly and the administering subcutaneously can each independently comprise a dosage of about 90 mg of the GnRH antagonist or salt thereof.

Also disclosed herein are methods of administering a GnRH antagonist or salt thereof to a subject, where the administration can result in a PK profile substantially as depicted in FIG. 8 after a first administration comprising an administering intramuscularly and an administering subcutaneously of the GnRH antagonist or salt thereof to the subject; followed by a second administration comprising an administering subcutaneously of the GnRH antagonist or salt thereof to the subject; where the second administration can be performed about 28 days after the first administration.

In some instances, the first administration can comprise administering intramuscularly a dose of about 90 mg of the GnRH antagonist or salt thereof. In some instances, the first administration can comprise administering subcutaneously a dose of about 90 mg of the GnRH antagonist or salt thereof. In some instances, the first administration can comprise administering intramuscularly a dose of about 90 mg of the GnRH antagonist or salt thereof and administering subcutaneously a dose of about 90 mg of the GnRH antagonist or salt thereof. In some instances, the second administration can comprise administering subcutaneously a dose of about 90 mg of the GnRH antagonist or salt thereof.

In some instances, the GnRH antagonist or salt thereof can be teverelix or a salt thereof. In some instances, the salt of the GnRH antagonist can be teverelix trifluoroacetate.

In some instances, the GnRH antagonist or salt thereof can be a peptide or salt thereof. In some instances, the GnRH antagonist or salt thereof can comprise a crystalline form, where the crystalline form can be in suspension in a liquid. In some instances, the crystalline form can be microcrystalline.

In some instances, a salt of the GnRH antagonist can comprise a counter-ion, where the counter-ion can be the conjugate base of an acid having a pKa of less than about 2. In some instances, the counter-ion can be the conjugate base of sulfuric acid, trifluoromethanesulfonic acid, trifluoroacetic acid, or benzenesulfonic acid.

In some instances, the GnRH antagonist or salt thereof individually can be abarelix, cetrorelix, degarelix, ganirelix, ozarelix, antide, teverelix, or a salt of any of these. In some instances, the GnRH antagonist or salt thereof can be teverelix or a salt thereof. In some instances, the salt of the GnRH antagonist can be teverelix trifluoroacetate.

Also disclosed herein are methods comprising: (a) administering intramuscularly a therapeutically effective amount of a first GnRH antagonist or a salt thereof to a subject, and (b) administering subcutaneously a therapeutically effective amount of a second GnRH antagonist or a salt thereof to the subject, where the administering of the first GnRH antagonist or salt thereof and the administering of the second GnRH antagonist or salt thereof can be performed within about 168 hours of one another.

In some instances, the administering of the first GnRH antagonist or salt thereof and the administering of the second GnRH antagonist or salt thereof can be performed within 24 hours of one another.

In some instances, the administering intramuscularly can be performed before the administering subcutaneously. In some instances, the administering subcutaneous can be performed before the administering intramuscularly. In some instances, the administering intramuscularly and the administering subcutaneously can be performed substantially simultaneously. In some instances, the administering intramuscularly and the administering subcutaneously independently comprise an injection.

In some instances, the first GnRH antagonist or salt thereof and the second GnRH antagonist or salt thereof can be the same. In some instances, the first GnRH antagonist or salt thereof and the second GnRH antagonist or salt thereof can be different.

In some instances, the method can be a method of at least partially ameliorating a disease or condition. In some instances, the disease or condition can be an androgen-sensitive condition. In some instances, the disease or condition can be benign prostatic hyperplasia, acute urinary retention, a cancer, or any combination thereof. In some instances, the disease or condition may not be benign prostatic hyperplasia, acute urinary retention, a cancer, or any combination thereof.

In some instances, the disease or condition can be a cancer that can be prostate cancer.

In some instances, prostate size can be reduced in the subject after the administering intramuscularly and the administering subcutaneously. In some instances, the prostate size can be reduced by at least about 10% by volume as measured by ultrasound within 12 weeks after the administering intramuscularly and the administering subcutaneously, relative to a prostate size prior to the administering intramuscularly and the administering subcutaneously. In some instances, the prostate size can be reduced by at least about 10% by mass as measured by ultrasound within 12 weeks after the administering intramuscularly and the administering subcutaneously, relative to a prostate size prior to the administering intramuscularly and the administering subcutaneously.

In some instances, urine flow can be increased in the subject after the administering intramuscularly and the administering subcutaneously. In some instances, the urine flow can be increased by at least about 10% as measured by an electronic uroflowmeter within 4 weeks after the administering intramuscularly and the administering subcutaneously relative to a urine flow prior to the administering intramuscularly and the administering subcutaneously.

In some instances, the method can be a method of reducing prostate size in the subject. In some instances, the prostate size can be reduced by at least about 10% by mass or volume as measured by ultrasound within 12 weeks after the administering intramuscularly and the administering subcutaneously, relative to a prostate size prior to the administering intramuscularly and the administering subcutaneously.

In some instances, the method can be a method of chemical castration.

In some instances, the subject can be a primate. In some instances, the subject can be a canine or a feline. In some instances, the subject can be human. In some instances, the subject can be a male. In some instances, the subject can be a female. In some instances, the subject can be in need thereof.

In some instances, the method can be a method of reducing blood plasma testosterone level in the subject, where the blood plasma testosterone level can be reduced relative to a blood plasma testosterone level before treatment with the method. In some instances, the testosterone can be elevated as a result of hyperthyroidism, Grave's disease, precocious puberty, or cancer.

In some instances, the method can be a method of treating hyperthyroidism, Grave's disease, precocious puberty, or cancer.

In some instances, the method can be a method of birth control.

In some instances, the administering intramuscularly and the administering subcutaneously provide a blood plasma testosterone concentration less than about: 3 ng/mL, 2.5 ng/mL, 2 ng/mL, 1.5 ng/mL, 1 ng/mL, or 0.5 ng/mL as measured by mass spectroscopy. In some instances, the administering intramuscularly and the administering subcutaneously provide a blood plasma testosterone concentration less than about 0.5 ng/mL. In some instances, a blood plasma testosterone concentration of less than about 0.5 ng/mL can be achieved by about: 24, 48, or 72 hours after the administering subcutaneously and the administering intramuscularly.

In some instances, the method can further comprise an additional administering of a GnRH antagonist or salt thereof. In some instances, the additional administering can be performed at least about 28 or more days after the administering intramuscularly and the administering subcutaneously. In some instances, a blood plasma testosterone concentration in the subject can be maintained below at least about 0.5 ng/mL from about: 24, 48, or 72 hours after the administering intramuscularly and the administering subcutaneously until the additional administering.

In some instances, the method can further comprise administering the GnRH antagonist or salt thereof at a dosing interval of at least about 28 days. In some instances, a blood plasma testosterone concentration in the subject can be substantially maintained below about 2 ng/mL throughout the dosing interval. In some instances, a blood plasma testosterone concentration in the subject can be substantially maintained below about 0.5 ng/mL throughout the dosing interval.

In some instances, the method can further comprise reducing the subject's blood plasma prostate specific antigen (PSA) concentration to less than about 4 ng/mL.

In some instances, the method can be a method of treating breast cancer.

In some instances, the disease or condition can be an estrogen-sensitive condition. In some instances, the method can be a method of reducing a blood plasma estrogen level in the subject, where the blood plasma estrogen level can be reduced relative to a blood plasma estrogen level before treatment with the method. In some instances, the estrogen can be an estradiol.

In some instances, the method can be a method reducing pain. In some instances, the pain can be associated with endometriosis.

In some instances, the first GnRH antagonist or salt thereof and the second GnRH antagonist or salt thereof can be independently a peptide or salt thereof. In some instances, the first GnRH antagonist or salt thereof and the second GnRH antagonist or salt thereof can be independently in a crystalline form, where the crystalline form can be in suspension in a solution. In some instances, the first GnRH antagonist or salt thereof and the second GnRH antagonist or salt thereof independently comprise a microcrystalline peptide in suspension.

In some instances, the first GnRH antagonist salt and the second GnRH antagonist salt can be independently a peptide salt comprising a counter-ion, where the counter-ion can be the conjugate base of an acid having a pKa of less than about 2. In some instances, the counter-ion can be the conjugate base of sulfuric acid, trifluoromethanesulfonic acid, trifluoroacetic acid, or benzenesulfonic acid.

In some instances, the first GnRH antagonist or salt thereof and the second GnRH antagonist or salt thereof can be independently abarelix, cetrorelix, degarelix, ganirelix, ozarelix, antide, teverelix, or a salt of any of the above. In some instances, the first GnRH antagonist or salt thereof and the second GnRH antagonist or salt thereof can be independently teverelix or a salt thereof. In some instances, the first GnRH antagonist or salt thereof and the second GnRH antagonist or salt thereof can be independently teverelix trifluoroacetate.

In some instances, the administering intramuscularly and the administering subcutaneously independently can comprise administering a dosage ranging from about 30 mg to about 240 mg of teverelix trifluoroacetate. In some instances, the administering can comprise a dosage of about 90 mg of teverelix trifluoroacetate.

In some instances, the method can further comprise providing a blood plasma teverelix concentration in the subject of at least about 9 ng/mL. In some instances, the method can further comprise providing a blood plasma Cmax of teverelix in the subject of at least about 19 ng/mL. In some instances, the method can further comprise providing a AUC₍₀₋ₜ₎ of teverelix in the subject of at least about 1,000 ng*h/mL, where t can be at least about 28 days after the first administering and the second administering. In some instances, the method can further comprise maintaining a blood plasma teverelix concentration in the subject above at least about 5 ng/mL from about: 24, 48, or 72 hours after the administering intramuscularly and the administering subcutaneously until the additional administering.

In some instances, the subject can be diagnosed with, previously diagnosed with, or can be suspected of having a disease or condition. In some instances, the disease or condition can be a cancer.

In some instances, the method can further comprise administering an additional pharmaceutical substance.

In some instances, the additional pharmaceutical substance can be an antineoplastic. In some instances, the antineoplastic can be cyclophosphamide, methotrexate, 5-fluorouracil, doxorubicin, procarbazine, prednisolone, bleomycin, vinblastine, dacarbazine, cisplatin, epirubicin, a salt of any of these, or any combination thereof.

In some instances, the additional pharmaceutical substance can be an adrenergic receptor antagonist. In some instances, the adrenergic receptor antagonist can be an alpha-blocker. In some instances, the adrenergic receptor antagonist can be a beta-blocker.

In some instances, the additional pharmaceutical substance can be phenoxybenzamine, phentolamine, tolazoline, trazodone, alfuzosin, doxazosin, prazosin, tamsulosin, terazosin, silodosin, carvedilol, labetalol, a salt of any of these, or any combination thereof. In some instances, the additional pharmaceutical substance can be propranolol, bucindolol, carteolol, carvedilol, labetalol, nadolol, oxprenolol, penbutolol, pindolol, sotalol, timolol, acebutolol, atenolol, betaxolol, bisoprolol, celiprolol, esmolol, metoprolol, nebivolol, butazamine, a salt of any of these, or any combination thereof.

In some instances, the additional pharmaceutical substance can be a 5α-reductase inhibitor. In some instances, the 5α-reductase inhibitor can be dutasteride, tamsulosin, finasteride, episteride, alfatradiol, a salt of any of these, or any combination thereof.

In some instances, the first GnRH antagonist or salt thereof and the second GnRH antagonist or salt thereof independently can comprise GnRH antagonist crystals suspended in solution. In some instances, the first GnRH antagonist or salt thereof and the second GnRH antagonist or salt thereof can independently comprise a microcrystalline aqueous suspension comprising teverelix trifluoroacetate. In some instances, the microcrystalline aqueous suspension comprising teverelix trifluoroacetate when lyophilized can be stable for at least a year when stored in a sealed container at a temperature of from about 2° C. to about 8° C. at 50% relative humidity as measured by a retention of α-helical content after storage compared to prior to storage, where the α-helical content can be determined by circular dichroism.

In some instances, the first GnRH antagonist or salt thereof and the second GnRH antagonist or salt thereof can be in a formulation further comprising an isotonic agent. In some instances, the isotonic agent can be a polyhedric alcohol. In some instances, the polyhedric alcohol can be a sugar alcohol. In some instances, the isotonic agent can be mannitol.

In some instances, at least one of the first GnRH antagonist or salt thereof; or the second GnRH antagonist or salt thereof can be in a formulation further comprising a pharmaceutically acceptable excipient.

Also disclosed herein are methods for preparing a microcrystalline aqueous suspension described herein, comprising contacting a microcrystalline substance with a solution.

Also disclosed herein are methods of chemical castration, comprising: (a) administering intramuscularly of teverelix trifluoroacetate to a subject; and (b) administering subcutaneously of teverelix trifluoroacetate to the subject, where the administering intramuscularly and the administering subcutaneously can be performed within about 24 hours, and where a blood plasma testosterone concentration in the subject of less than about 0.5 ng/mL can be achieved by about 48 hours after the administering intramuscularly and the administering subcutaneously.

Also disclosed herein are kits comprising at least one GnRH antagonist or salt thereof described herein, and instructions for use thereof.

In some instances, the instructions for use can provide instructions for treating a disease or condition, or a symptom thereof. In some instances, the instructions can describe administering the GnRH antagonist or salt thereof to produce chemical castration.

In some instances, the kit can further comprise a syringe.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of embodiments are set forth with particularity in the appended claims. A better understanding of the features and advantages of the embodiments will be obtained by reference to the following detailed description that sets forth illustrative embodiments, in which the principles of the embodiments are utilized, and the accompanying drawings of which:

FIG. 1 depicts a testosterone and teverelix time course after i.m. administration.

FIG. 2A depicts a mean concentration of teverelix over time following three s.c. 90 mg doses of teverelix.

FIG. 2B depicts a mean concentration of teverelix over time following two i.m. 90 mg doses of teverelix.

FIG. 3A depicts a mean concentration of PSA over time following three s.c. 90 mg doses of teverelix.

FIG. 3B depicts a mean concentration of PSA over time following two i.m. 90 mg doses of teverelix.

FIG. 4 depicts an extrapolated mean concentration of teverelix over time following a single i.m. 90 mg dose of teverelix.

FIG. 5 depicts an extrapolated mean concentration of teverelix over time following a single s.c. 90 mg dose of teverelix.

FIG. 6 depicts a simulated PK profile of a GnRH antagonist after a 90 mg i.m.+90 mg s.c. administration, followed by a 90 mg s.c. dosage every 56 days.

FIG. 7 depicts a simulated PK profile of a GnRH antagonist after a 90 mg i.m.+90 mg s.c. administration, followed by a 90 mg s.c. dosage every 42 days.

FIG. 8 depicts a simulated PK profile of a GnRH antagonist after a 90 mg i.m.+90 mg s.c. administration, followed by a 90 mg s.c. dosage every 28 days.

FIG. 9 depicts a PK profile of teverelix TFA after a 90 mg i.m.+90 mg s.c. administration, followed by a 90 mg s.c. dosage every 56 days.

FIG. 10 depicts a PK profile teverelix TFA after a 90 mg i.m.+90 mg s.c. administration, followed by a 90 mg s.c. dosage every 42 days.

FIG. 11 depicts a PK profile teverelix TFA after a 90 mg i.m.+90 mg s.c. administration, followed by a 90 mg s.c. dosage every 28 days.

### DETAILED DESCRIPTION

### I. Overview

Described herein are methods of administering a GnRH antagonist with the surprising and unexpected result of dramatically improved pharmacokinetics. Such improvements can be utilized for the treatment of conditions or diseases through a suppression of aberrantly-expressed hormones.

Disclosed herein are methods comprising administering to a subject in need thereof a therapeutically effective amount of a GnRH antagonist or a salt thereof, where the administering of the GnRH antagonist or salt thereof can produce a Cmax of at least about 19 ng/mL and an AUC(0-t) from about 1,000 ng*h/mL to about 3,000 ng*h/mL when administered at a total dose of about 240 mg and a dosing interval of about 28 days.

Also disclosed herein are methods comprising: (a) an intramuscular administering of a therapeutically effective amount of a first GnRH antagonist or a salt thereof to a subject, (b) a subcutaneous administering of a therapeutically effective amount of a second GnRH antagonist or a salt thereof to the subject, where the administering of a first GnRH antagonist or salt thereof and the administering of a second GnRH antagonist or salt thereof are performed within 168 hours. A dual administration as described herein can produce a surprising and unexpected prolonging of the circulatory half-life of the GnRH antagonist relative to the individual injections alone, thereby prolonging the duration of a therapeutic effect of the GnRH antagonist in vivo.

Also disclosed herein are methods of at least partially ameliorating a disease or condition comprising administering to a subject a GnRH antagonist. In some exemplary embodiments, the method can be used to at least partially ameliorate a condition such as prostate cancer through a suppression of gonadotropins such as testosterone and dihydrotestosterone (DHT).

Also disclosed herein are kits comprising at least one GnRH antagonist and instructions for use. A kit described herein can comprise means to perform a dual injection as described herein, as well as instructions for performing the dual injection.

### II. Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, i.e., the limitations of the measurement system. For example, “about” can mean about plus or minus 10%, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “subject”, “patient” or “individual” as used herein in reference to an individual, and can encompass a mammal and a non-mammal. A mammal can be any member of the Mammalian class, including but not limited to a human; a non-human primates such as a chimpanzee, an ape or other monkey species; a farm animal such as cattle, a horse, a sheep, a goat, a swine; a domestic animal such as a rabbit, a dog (or a canine), and a cat (or a feline); or a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, the human can be an adult. In some instances, the human can be a child. In some instances, the human can be age 0-17 years old. In some instances, the human can be age 18-130 years old. In some instances, the subject can be a male. In some cases, a male can comprise a subject born with at least a portion of a prostate. In some cases, a male can comprise a subject born with a Y chromosome. In some instances, a male can comprise a subject born with male gonads. In some instances, the subject can be a female. In some cases, a female can comprise a subject born without at least a portion of a prostate. In some cases, a female can comprise a subject born without a Y chromosome. In some instances, a female can comprise a subject born with female gonads. In some instances, a female can comprise a subject born with an ovary. In some instances, the subject is diagnosed with, or is suspected of having, a condition or disease such as cancer. In some instances, the subject can be a sexual offender. In some instances, the subject can be a pedophile. In some instances, the subject can have at least a portion of a prostate. In some cases, the subject can have at least a portion of an endometrium.

The terms “treat,” “treating”, “treatment,” “ameliorate” or “ameliorating” and other grammatical equivalents as used herein, can include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms can further include achieving a therapeutic benefit and/or a prophylactic benefit. Therapeutic benefit can mean eradication or amelioration of the underlying disease being treated. Also, a therapeutic benefit can be achieved with the eradication or amelioration of one or more of the physiological symptoms associated with the underlying disease such that an improvement can be observed in the patient, notwithstanding that, in some embodiments, the patient can still be afflicted with the underlying disease.

The terms “effective amount”, “therapeutically effective amount” or “pharmaceutically effective amount” as used herein, can refer to a sufficient amount of a compound being administered which will at least partially ameliorate a symptom of a disease or condition being treated.

The terms “co-administration”, “administered in combination with” and their grammatical equivalents or the like, as used herein, can encompass administration of selected therapeutic agents to a single subject, and can include treatment regimens in which the agents are administered by the same or different route of administration or at the same or different times. In some embodiments, a compound disclosed herein can be co-administered with other agents. These terms can encompass administration of two or more agents to a subject so that both agents and/or their metabolites are present in the subject at the same time. They can include simultaneous administration, administration at different times, and/or administration in a composition in which both agents are present. Thus, in some embodiments, a compound and another agent(s) can be administered in a single composition. In some embodiments, a compound and another agent(s) can be admixed in the composition. In some embodiments, the same compound can be administered via a combination of different routes of administration.

As used herein, the term “bioavailability” can denote the degree to which a drug or other substance becomes available to the target tissue after administration.

Parameters often used in pharmacokinetic (PK) studies can include Tmax, Cmax, AUC(0-∞), AUC(0-t), and T_{1/2} and CL/F. “Tmax” can refer to the time to reach the maximal plasma concentration (“Cmax”) after administration of a therapeutic; “AUC(0-00)” can refer to the area under the plasma concentration versus time curve from time 0 to infinity; “AUC(0-t)” can refer to the area under the plasma concentration versus time curve from time 0 to time t; “T_{1/2}” can refer to a half-life of a therapeutic in blood plasma; “T_{1/2, elim}” can refer to the half-life of elimination of the therapeutic from circulation; and “CL/F” can refer to an apparent clearance rate of a therapeutic.

As used herein, the term “gonadotropin releasing hormone antagonist” can be understood to include a “gonadotropin releasing hormone receptor antagonist.”

### III. Gonadotropin Releasing Hormone Antagonist Formulations

Disclosed herein are methods of improving the pharmacokinetic (PK) profile/pharmacodynamics (PD) of a therapeutic or salt thereof in vivo.

In some instances, the therapeutic can be a gonadotropin-releasing hormone (GnRH) antagonist or a salt thereof. Gonadotropin-releasing hormone antagonists (GnRH antagonists) are synthetic peptides that compete with the endogenous neurohormone GnRH (otherwise known as luteinizing hormone releasing hormone, LHRH) for binding to its receptors in the anterior pituitary gland. By decreasing or blocking GnRH action, GnRH antagonists suppress release from the anterior pituitary gland of gonadotropins such as follicle stimulating hormone (FSH) and luteinizing hormone (LH).

Both FSH and LH are involved in normal reproductive function. In females, FSH stimulates the growth of immature Graafian follicles to maturation, whereas changes in LH levels control ovulation. In males, on the other hand, FSH plays an important role in spermatogenesis and LH stimulates production of testosterone in the testes.

Suppression of FSH and LH release can be used to treat a condition or disease mediated by the downstream action of the gonadotropins such as prostate cancer. This can involve surgical intervention such as surgical castration, which can be irreversible and invasive. Suppression can also be mediated through administration of a therapeutic designed to reduce the action of GnRH, and thereby suppress the downstream secretion of hormones.

One possible family of therapeutics are GnRH agonists such as leuprorelin, goserelin, triptorelin, buserelin, and histrelin. GnRH agonists have been shown to be a highly effective and safe alternative to surgical castration in the treatment of advanced prostate cancer. GnRH agonists work by overstimulating the GnRH receptor, thereby producing an initial burst of the gonadotropin. This overstimulation results in a negative feedback loop, which downregulates production of GnRH receptors, and thereby at least partially ameliorates the condition.

GnRH agonists, however, can have inherent drawbacks in their use. For instance, the administration of a GnRH agonist can produce an initial burst in the production of the gonadotropin, thereby potentially exacerbating the condition further. Furthermore, the time scale of suppression of the gonadotropin can be on the order of days, and the response to the agonist is typically not dose dependent. Administration of a GnRH agonist can also increase rick of cardiovascular disease, thereby offsetting any gains in safety produced by opting out of surgery.

GnRH antagonists are an alternative family of therapeutics to GnRH agonists, which can induce a rapid and reversible chemical castration without the initial stimulation (increase in secretion of gonadotropins and sex steroids) characteristics of GnRH agonists.

A GnRH antagonist can compete with natural gonadotropin releasing hormones for binding to pituitary receptors, which can suppress gonadotropins. Such suppression in men can reduce hormones in men such as testosterone and dihydrotestosterone. In some instances, such suppression obtained with a GnRH antagonist can be immediate. In exemplary short-term studies, GnRH antagonists appear to suppress LH to a considerably greater degree than FSH. However, exemplary long-term studies indicate suppression of both LH and FSH. In some instances, the duration of hormone suppression after administration of a GnRH antagonist can be dose-dependent in contrast to the use of a GnRH agonist. Furthermore, the use of a GnRH antagonist can produce a more rapid recovery after cessation of use than a GnRH agonist, and carries a lower overall risk of cardiovascular disease than a GnRH agonist.

In some cases, the GnRH antagonist or salt thereof can be abarelix, cetrorelix, degarelix, ganirelix, ozarelix, antide, teverelix, or a salt of any of these. A GnRH antagonist can be a salt thereof. In some instances, recitation of the phrase “GnRH antagonist” should be construed to include a salt thereof even if not explicitly recited.

In some instances, a salt can include a carboxylate salt (e.g. formate, acetate, trifluoroacetate, trichloroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, pamoate, nicotinate, isonicotinate, cinnamate, oxalate, malonate, succinate, suberate, sebacate, fumarate, malate, maleate, hydroxymaleate, hippurate, phthalate or a terephthalate salt); a halide salt (e.g. chloride, bromide or iodide salts); a sulfonate salt (e.g. benzene sulfonate, methyl-, bromo- or chloro-benzenesulfonate, xylenesulfonate, methanesulfonate, trifluoromethanesulfonate, ethanesulfonate, propanesulfonate, hydroxyethanesulfonate, 1- or 2-naphthalene-sulfonate or 1,5-naphthalenedisulfonate salts); a sulfate salt; a pyrosulfate salt; a bisulfate salt; a sulfite salt; a bisulfite salt; a phosphate salt; a monohydrogenphosphate salt; a dihydrogenphosphate salt; a metaphosphate salt; a pyrophosphate salt; a nitrate salt; and the like.

In some instances, a salt can be a pharmaceutically acceptable salt. In some instances, the pharmaceutically acceptable salt can be a salt described in Berge et al, J. Pharm. Sci, 1977. In some instances, a pharmaceutically acceptable salts can include those salts prepared by reaction of a GnRH antagonist with a mineral, organic acid or inorganic base, such salts including, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate and xylenesulfonate.

In some embodiments, the GnRH antagonist can comprise a counter ion that is a conjugate base of an acid. In some instances, the acid can have a pKa of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In some instances, the acid can have a pKa of less than about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In some instances, the acid can have a pKa of from about 1.0 to about 7.0, from about 1.5 to about 7.0, from about 2.0 to about 7.0, from about 2.5 to about 7.0, from about 3.0 to about 7.0, from about 3.5 to about 7.0, from about 4.0 to about 7.0, from about 4.5 to about 7.0, from about 5.0 to about 7.0, from about 5.5 to about 6.0, or from about 6.5 to about 7.0.

In some instances, the GnRH antagonist can be teverelix ((Ac-D-NaID-pClPhe-D-Pal-Ser-Tyr-D-Hci-Leu-Lys(iPr)-Pro-D-Ala-NH₂). Teverelix is a synthetic decapeptide with a water solubility of approximately 10 mg/mL.

Formulations comprising many counter ions of teverelix can form gels. However, it is advantageous to formulate and administer a solution or microcrystalline suspension of teverelix, as such formulations can provide both immediate and prolonged release depending on the specific formulation. WO 2003/022243 discloses production of strong acid salts of teverelix, such as triflouroacetic acid and sulfulic acid. Such salts can provide release of teverelix over a prolonged period of time. Alternatively, administration of the acetate salt of teverelix can provide immediate release when administered subcutaneously. In some exemplary embodiments, the GnRH salt administered is teverelix trifluoromethanesulfonate, teverelix triflouroacetate, teverelix benesulfonate, teverelix acetate, or a combination thereof.

Teverelix TFA has inherent advantages over other GnRH antagonists, including a lower risk of local irritation or anaphylactic reactions. For example, teverelix has been shown to produce less local irritation at the administration site than degarelix, as well as display of a more dose-dependent response to administration.

In some instances, a GnRH antagonist can be peptide. In some cases, a GnRH antagonist can be in a crystalline form. In some instances, a GnRH antagonist can be in a microcrystalline form. In some instances, a GnRH antagonist can be in the form of a solution, an emulsion, a gel, or a suspension. In some exemplary embodiments, a GnRH antagonist can comprise a crystalline suspension. In some exemplary embodiments, a GnRH antagonist can comprise a microcrystalline peptide suspension.

Also disclosed herein are methods of preparing a GnRH antagonist or a salt thereof as a microcrystalline aqueous suspension comprising contacting a microcrystalline form of the GnRH antagonist with an aqueous solution or diluent.

In some cases, the GnRH antagonist can be formulated as a composition comprising an excipient, diluent, or carrier. In some cases, the composition can be a pharmaceutical composition comprising a pharmaceutically acceptable excipient, diluent, or carrier.

In some embodiments, the diluent can be water or an aqueous buffering agent. In some embodiments, the buffering agent can be saline. In some embodiments, the buffering agent can be citrate. In some embodiments, the buffering agent can be phosphate. In some embodiments, the buffering agent can be acetate. In some embodiments, the buffering agent can be glycine. In some embodiments, the buffering agent can be tris(hydroxymethyl)aminomethane (tris). In some embodiments, the buffering agent can be 4-(2-hydroxyethyl)-1-piperazineethanesulfonic acid (HEPES). In some embodiments, the buffering agent can be piperazine-N,N′-bis(2-ethanesulfonic acid) (PIPES).

In some cases, the composition can comprise an isotonic agent. In some embodiments, the isotonic agent can be a polyhedric alcohol or a polyol. In some embodiments, the polyol does not comprise ethylene glycol. In some embodiments, the polyol can be propylene glycol. In some embodiments, the polyol can be a sugar. In some embodiments, the sugar can be sucrose. In some embodiments, the sugar can be glucose. In some embodiments, the sugar can be fructose. In some embodiments, the sugar can be maltose. In some embodiments, the polyol can be a sugar alcohol. In some embodiments, the sugar alcohol can be glycerol. In some embodiments, the sugar alcohol can be erythritol. In some embodiments, the sugar alcohol can be threitol. In some embodiments, the sugar alcohol can be arabitol. In some embodiments, the sugar alcohol can be xylitol. In some embodiments, the sugar alcohol can be ribitol. In some embodiments, the sugar alcohol can be sorbitol. In some embodiments, the sugar alcohol can be galactitol. In some embodiments, the sugar alcohol can be fucitol. In some embodiments, the sugar alcohol can be iditol. In some embodiments, the sugar alcohol can be inositol. In some embodiments, the sugar alcohol can be volemitol. In some embodiments, the sugar alcohol can be isomalt. In some embodiments, the sugar alcohol can be maltitol. In some embodiments, the sugar alcohol can be lactitol. In some embodiments, the sugar alcohol can be maltotriitol. In some embodiments, the sugar alcohol can be mannitol. In some embodiments, the sugar alcohol can be maltotetraitol. In some embodiments, the sugar alcohol can be polyglycitol.

In some exemplary embodiments, an aqueous suspension comprising a GnRH antagonist can be lyophilized. In some instances, the lyophilized formulation is stable for at least about 1 month, 2 months, 3 months, 4 months, 5 months, 6 months, 7 months, 8 months, 9 months, 10 months, 11 months, 1 year, 2 years, 3 years, 4 years, or 5 years when stored in a closed container at 10%, 20%, 30%, 40%, 50%, 60%, 70%, 80%, or 90% relative humidity at a temperature of from about 2° C. to about 30° C., from about 2° C. to about 29° C., from about 2° C. to about 28° C., from about 2° C. to about 27° C., from about 2° C. to about 26° C., from about 2° C. to about 25° C., from about 2° C. to about 24° C., from about 2° C. to about 23° C., from about 2° C. to about 22° C., from about 2° C. to about 21° C., from about 2° C. to about 20° C., from about 2° C. to about 19° C., from about 2° C. to about 18° C., from about 2° C. to about 17° C., from about 2° C. to about 16° C., from about 2° C. to about 15° C., from about 2° C. to about 14° C., from about 2° C. to about 13° C., from about 2° C. to about 12° C., from about 2° C. to about 11° C., from about 2° C. to about 10° C., from about 2° C. to about 9° C., from about 2° C. to about 8° C., from about 2° C. to about 7° C., from about 2° C. to about 6° C., from about 2° C. to about 5° C., from about 2° C. to about 4° C., or from about 2° C. to about 3° C.

A GnRH described herein can also be formulated with a microparticle. In some cases, the microparticle can be a microsphere such as a hollow microsphere used as a carrier for the therapeutic. In some instances, a GnRH antagonist can be formulated with a nanoparticle. In some instances, the microparticle or nanoparticle can be a plastic, a metal, or a polymer.

### IV. Dosing/Administration

Disclosed herein are methods of enhancing the PK/PD profile of an administered GnRH antagonist. In some instances, a first GnRH antagonist can be administered to a subject through a first route of administration. In some instances, a second GnRH antagonist can be administered to a subject through a second route of administration. In embodiments, the administration of the first and second GnRH antagonist via the first and second route of administration, respectively, can be employed to improve the PK/PD profile of the first and second GnRH antagonist.

In some cases, the first GnRH antagonist and the second GnRH antagonist can be the same. In some cases, the first GnRH antagonist and the second GnRH antagonist can be different.

In some cases, the administration of the first GnRH antagonist and the second GnRH antagonist are performed simultaneously. In some cases, the administration of the first GnRH antagonist and the second GnRH antagonist can be separated by a period of time.

In some cases, the period of separation can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. In some cases, the period of separation can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes. In some cases, the period of separation can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, or 240 hours. In some cases, the period of separation can be at least about 1, 2, 3, 4, 5, 6, 7, 8, 9, 10, 11, 12, 13, or 14 days.

In some instances, the period of separation can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. In some instances, the period of separation can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes. In some cases, the period of separation can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, or 240 hours. In some cases, the period of separation can be no more than about 1, 2, 3, 4, 5, 6, 7, 8, 9, 10, 11, 12, 13, or 14 days.

In some instances, a GnRH antagonist or salt thereof described herein can be administered as a treatment course comprising a defined dosing frequency or a dosing interval. In some cases, the administration can be at a dosing frequency of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 times a month. In some instances, a GnRH antagonist or salt thereof described herein can be administered at a dosing interval of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 days apart.

In some instances, the first GnRH antagonist can be administered before the second GnRH antagonist. In some instances, the second GnRH antagonist can be administered before the first GnRH antagonist.

Administration of a GnRH antagonist described herein can be performed via any type of administration route known in the art. Examples can include injection or infusion, (including intra-arterial, intracardiac, intracerebroventricular, intradermal, intraduodenal, intramedullary, intramuscular (i.m.), intraosseous, intraperitoneal, intrathecal, intravascular, intravenous, intravitreal, epidural, and subcutaneous (s.c.)), inhalational, transdermal, transmucosal, sublingual, buccal and topical (including epicutaneous, dermal, enema, eye drops, ear drops, intranasal, and vaginal) administration. In some exemplary embodiments, the route of administration can be via an injection such as an intramuscular, intravenous, subcutaneous, or intraperitoneal injection.

In some exemplary embodiments, a GnRH antagonist or salt thereof can be administered via an intramuscular administration. In some exemplary embodiments, a GnRH antagonist can be administered via a subcutaneous administration.

In some exemplary embodiments, a first GnRH antagonist can be administered via an intramuscular administration and a second GnRH antagonist can be administered via a subcutaneous administration. Such an administration can produce a surprising and unexpected increase in useful PK parameters such as a T_{1/2} of the GnRH in circulation, which can produce a physiological result such as an increase in the duration of chemical castration brought about through administration of the GnRH antagonist.

In some cases, a GnRH antagonist or salt thereof described herein can be administered at a dose of from about 1 mg to about 480 mg, from about 5 mg to about 480 mg, from about 10 mg to about 480 mg, from about 15 mg to about 480 mg, from about 20 mg to about 480 mg, from about 25 mg to about 480 mg, from about 30 mg to about 480 mg, from about 35 mg to about 480 mg, from about 40 mg to about 480 mg, from about 45 mg to about 480 mg, from about 50 mg to about 480 mg, from about 55 mg to about 480 mg, from about 60 mg to about 480 mg, from about 65 mg to about 480 mg, from about 70 mg to about 480 mg, from about 75 mg to about 480 mg, from about 80 mg to about 480 mg, from about 85 mg to about 480 mg, from about 90 mg to about 480 mg, from about 95 mg to about 480 mg, from about 100 mg to about 480 mg, from about 105 mg to about 480 mg, from about 110 mg to about 480 mg, from about 115 mg to about 480 mg, from about 120 mg to about 480 mg, from about 125 mg to about 480 mg, from about 130 mg to about 480 mg, from about 135 mg to about 480 mg, from about 140 mg to about 480 mg, from about 145 mg to about 480 mg, from about 150 mg to about 480 mg, from about 155 mg to about 480 mg, from about 160 mg to about 480 mg, from about 165 mg to about 480 mg, from about 170 mg to about 480 mg, from about 175 mg to about 480 mg, from about 180 mg to about 480 mg, from about 185 mg to about 480 mg, from about 190 mg to about 480 mg, from about 195 mg to about 480 mg, from about 200 mg to about 480 mg, from about 205 mg to about 480 mg, from about 210 mg to about 480 mg, from about 215 mg to about 480 mg, from about 220 mg to about 480 mg, from about 225 mg to about 480 mg, from about 230 mg to about 480 mg, from about 235 mg to about 480 mg, from about 240 mg to about 480 mg, from about 245 mg to about 480 mg, from about 250 mg to about 480 mg, from about 255 mg to about 480 mg, from about 260 mg to about 480 mg, from about 265 mg to about 480 mg, from about 270 mg to about 480 mg, from about 275 mg to about 480 mg, from about 280 mg to about 480 mg, from about 285 mg to about 480 mg, from about 290 mg to about 480 mg, from about 295 mg to about 480 mg, or from about 300 mg to about 480 mg. In some cases, a GnRH antagonist or salt thereof described herein can be administered at a dose of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 242, 243, 244, 245, 246, 247, 248, 249. 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, or 480 mg.

In some cases, a GnRH antagonist or salt thereof described herein can be administered to provide a blood plasma concentration of the GnRH antagonist, metabolite, or salt thereof of from about 0.5 ng/mL to about 10 ng/mL, from about 1 ng/mL to about 10 ng/mL, from about 1.5 ng/mL to about 10 ng/mL, from about 2 ng/mL to about 10 ng/mL, from about 2.5 ng/mL to about 10 ng/mL, from about 3 ng/mL to about 10 ng/mL, from about 3.5 ng/mL to about 10 ng/mL, from about 4 ng/mL to about 10 ng/mL, from about 4.5 ng/mL to about 10 ng/mL, from about 5 ng/mL to about 10 ng/mL, from about 5.5 ng/mL to about 10 ng/mL, from about 6 ng/mL to about 10 ng/mL, from about 6.5 ng/mL to about 10 ng/mL, from about 7 ng/mL to about 10 ng/mL, from about 7.5 ng/mL to about 10 ng/mL, from about 8 ng/mL to about 10 ng/mL, from about 8.5 ng/mL to about 10 ng/mL, from about 9 ng/mL to about 10 ng/mL, or from about 9.5 ng/mL to about 10 ng/mL.

In some cases, a GnRH antagonist or salt thereof described herein can be administered to provide a blood plasma concentration of the GnRH antagonist, metabolite, or salt thereof after administration to a subject of at least about 20 ng/mL, 19.5 ng/mL, 19 ng/mL, 18.5 ng/mL, 18 ng/mL, 17.5 ng/mL, 17 ng/mL, 16.5 ng/mL, 16 ng/mL, 15.5 ng/mL, 15 ng/mL, 14.5 ng/mL, 14 ng/mL, 13.5 ng/mL, 13 ng/mL, 12.5 ng/mL, 12 ng/mL, 11.5 ng/mL, 11 ng/mL, 10.5 ng/mL, 10 ng/mL, 9.5 ng/mL, 9 ng/mL, 8.5 ng/mL, 8 ng/mL, 7.5 ng/mL, 7 ng/mL, 6.5 ng/mL, 6 ng/mL, 5.5 ng/mL, 5 ng/mL, 4.5 ng/mL, 4 ng/mL, 3.5 ng/mL, 3 ng/mL, 2.5 ng/mL, 2 ng/mL, 1.5 ng/mL, 1 ng/mL, or 0.5 ng/mL.

In some cases, a GnRH antagonist or salt thereof described herein can be administered to provide a Cmax of the GnRH antagonist, metabolite, or salt thereof after administration to a subject of at least about 100 ng/mL, 95 ng/mL, 90 ng/mL, 85 ng/mL, 80 ng/mL, 75 ng/mL, 70 ng/mL, 65 ng/mL, 60 ng/mL, 55 ng/mL, 50 ng/mL, 45 ng/mL, 40 ng/mL, 35 ng/mL, 30 ng/mL, 25 ng/mL, 20 ng/mL, 15 ng/mL, 10 ng/mL, or 5 ng/mL. In some cases, a GnRH antagonist or salt thereof described herein can be administered to provide a Cmax of the GnRH antagonist, metabolite, or salt thereof of at least about 50 ng/mL, 49 ng/mL, 48 ng/mL, 47 ng/mL, 46 ng/mL, 45 ng/mL, 44 ng/mL, 43 ng/mL, 42 ng/mL, 41 ng/mL, 40 ng/mL, 39 ng/mL, 38 ng/mL, 37 ng/mL, 36 ng/mL, 35 ng/mL, 34 ng/mL, 33 ng/mL, 32 ng/mL, 31 ng/mL, 30 ng/mL, 29 ng/mL, 28 ng/mL, 27 ng/mL, 26 ng/mL, 25 ng/mL, 24 ng/mL, 23 ng/mL, 22 ng/mL, 21 ng/mL, 20 ng/mL, 19 ng/mL, 18 ng/mL, 17 ng/mL, 16 ng/mL, 15 ng/mL, 14 ng/mL, 13 ng/mL, 12 ng/mL, 11 ng/mL, 10 ng/mL, 9 ng/mL, 8 ng/mL, 7 ng/mL, 6 ng/mL, 5 ng/mL, 4 ng/mL, 3 ng/mL, 2 ng/mL, 1 ng/mL, or 0.5 ng/mL.

In some instances, a GnRH antagonist or salt thereof described herein can be administered to provide an AUC(0-t) of the GnRH antagonist, metabolite, or salt thereof after administration to a subject of at least about 10,000 ng*h/mL, 9,900 ng*h/mL, 9,800 ng*h/mL, 9,700 ng*h/mL, 9,600 ng*h/mL, 9,500 ng*h/mL, 9,400 ng*h/mL, 9,300 ng*h/mL, 9,200 ng*h/mL, 9,100 ng*h/mL, 9,000 ng*h/mL, 8,900 ng*h/mL, 8,800 ng*h/mL, 8,700 ng*h/mL, 8,600 ng*h/mL, 8,500 ng*h/mL, 8,400 ng*h/mL, 8,300 ng*h/mL, 8,200 ng*h/mL, 8,100 ng*h/mL, 8,000 ng*h/mL, 7,900 ng*h/mL, 7,800 ng*h/mL, 7,700 ng*h/mL, 7,600 ng*h/mL, 7,500 ng*h/mL, 7,400 ng*h/mL, 7,300 ng*h/mL, 7,200 ng*h/mL, 7,100 ng*h/mL, 7,000 ng*h/mL, 6,900 ng*h/mL, 6,800 ng*h/mL, 6,700 ng*h/mL, 6,600 ng*h/mL, 6,500 ng*h/mL, 6,400 ng*h/mL, 6,300 ng*h/mL, 6,200 ng*h/mL, 6,100 ng*h/mL, 6,000 ng*h/mL, 5,900 ng*h/mL, 5,800 ng*h/mL, 5,700 ng*h/mL, 5,600 ng*h/mL, 5,500 ng*h/mL, 5,400 ng*h/mL, 5,300 ng*h/mL, 5,200 ng*h/mL, 5,100 ng*h/mL, 5,000 ng*h/mL, 4,900 ng*h/mL, 4,800 ng*h/mL, 4,700 ng*h/mL, 4,600 ng*h/mL, 4,500 ng*h/mL, 4,400 ng*h/mL, 4,300 ng*h/mL, 4,200 ng*h/mL, 4,100 ng*h/mL, 4,000 ng*h/mL, 3,900 ng*h/mL, 3,800 ng*h/mL, 3,700 ng*h/mL, 3,600 ng*h/mL, 3,500 ng*h/mL, 3,400 ng*h/mL, 3,300 ng*h/mL, 3,200 ng*h/mL, 3,100 ng*h/mL, 3,000 ng*h/mL, 2,900 ng*h/mL, 2,800 ng*h/mL, 2,700 ng*h/mL, 2,600 ng*h/mL, 2,500 ng*h/mL, 2,400 ng*h/mL, 2,300 ng*h/mL, 2,200 ng*h/mL, 2,100 ng*h/mL, 2,000 ng*h/mL, 1,900 ng*h/mL, 1,800 ng*h/mL, 1,700 ng*h/mL, 1,600 ng*h/mL, 1,500 ng*h/mL, 1,400 ng*h/mL, 1,300 ng*h/mL, 1,200 ng*h/mL, 1,100 ng*h/mL, 1,000 ng*h/mL, 900 ng*h/mL, 800 ng*h/mL, 700 ng*h/mL, 600 ng*h/mL, 500 ng*h/mL, 400 ng*h/mL, 300 ng*h/mL, 200 ng*h/mL, or 100 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days after administration of the GnRH antagonist.

In some exemplary embodiments, a GnRH antagonist or salt thereof described herein can be administered to provide an AUC(0-t) of the GnRH antagonist, metabolite, or salt thereof after administration to a subject of from about 100 ng*h/mL to about 10,000 ng*h/mL, from about 100 ng*h/mL to about 9,000 ng*h/mL, from about 100 ng*h/mL to about 8,000 ng*h/mL, from about 100 ng*h/mL to about 7,000 ng*h/mL, from about 100 ng*h/mL to about 6,000 ng*h/mL, from about 100 ng*h/mL to about 5,000 ng*h/mL, from about 100 ng*h/mL to about 4,000 ng*h/mL, from about 100 ng*h/mL to about 3,000 ng*h/mL, from about 100 ng*h/mL to about 2,000 ng*h/mL, from about 100 ng*h/mL to about 100 ng*h/mL, from about 100 ng*h/mL to about 900 ng*h/mL, from about 100 ng*h/mL to about 800 ng*h/mL, from about 100 ng*h/mL to about 700 ng*h/mL, from about 100 ng*h/mL to about 600 ng*h/mL, from about 100 ng*h/mL to about 500 ng*h/mL, from about 100 ng*h/mL to about 400 ng*h/mL, from about 100 ng*h/mL to about 300 ng*h/mL, or from about 100 ng*h/mL to about 200 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days after administration of the GnRH antagonist.

In some exemplary embodiments, a GnRH antagonist or salt thereof described herein can be administered to provide an AUC(0-t) of the GnRH antagonist, metabolite, or salt thereof after administration to a subject of from about 1,000 ng*h/mL to about 10,000 ng*h/mL, from about 1,000 ng*h/mL to about 9,000 ng*h/mL, from about 1,000 ng*h/mL to about 8,000 ng*h/mL, from about 1,000 ng*h/mL to about 7,000 ng*h/mL, from about 1,000 ng*h/mL to about 6,000 ng*h/mL, from about 1,000 ng*h/mL to about 5,000 ng*h/mL, from about 1,000 ng*h/mL to about 4,000 ng*h/mL, from about 1,000 ng*h/mL to about 3,000 ng*h/mL, or from about 1,000 ng*h/mL to about 2,000 ng*h/mL, where t can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days after administration of the GnRH antagonist.

In some exemplary embodiments, a GnRH antagonist or salt thereof described herein can be administered to a subject to provide a blood plasma concentration in the subject of at least about 5 ng/mL for a time period of from about 6, 12, 18, 24, 30, 36, 42, 48, 54, 60, 66, 72, 78, 84, 90, 96, 102, 108, 114, or 120 hours after the administration.

In some instances, an additional pharmaceutical substance can be administered. In some instances, the additional pharmaceutical substance can be co-administered with the GnRH antagonist. In some instances, the additional pharmaceutical substance can be administered at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds apart. In some cases, the additional pharmaceutical substance can be administered at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes apart. In some cases, the additional pharmaceutical substance can be administered at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 184, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, or 240 hours apart. In some cases, the additional pharmaceutical substance can be administered at least about 1, 2, 3, 4, 5, 6, 7, 8, 9, 10, 11, 12, 13, or 14 days apart.

In some instances, the additional pharmaceutical substance can be an antineoplastic. Examples of antineoplastics can include cyclophosphamide, methotrexate, 5-fluorouracil, doxorubicin, procarbazine, prednisolone, bleomycin, vinblastine, dacarbazine, cisplatin, epirubicin, a salt of any of these, and any combination thereof.

In some instances, the additional pharmaceutical substance can be an adrenergic receptor antagonist such as an alpha blocker or a beta blocker. Examples of alpha blockers can include phenoxybenzamine, phentolamine, tolazoline, trazodone, alfuzosin, doxazosin, prazosin, tamsulosin, terazosin, silodosin, carvedilol, and labetalol. Examples of beta blockers can include propranolol, bucindolol carteolol, carvedilol, labetalol, nadolol, oxprenolol, penbutolol, pindolol, sotalol, timolol, acebutolol, atenolol, betaxolol, bisoprolol, celiprolol, esmolol, metoprolol, nebivolol, and butazamine.

In some instances, the additional pharmaceutical substance can be a 5α-reductase inhibitor. Examples of 5α-reductase inhibitors can include dutasteride, tamsulosin, finasteride, episteride, and alfatradiol. Additional examples can include herb and mineral-based inhibitors such as zinc; riboflavin (vitamin B2); azelaic acid; β-sitosterol; a polyphenol; alizarin; curcumin; a green tea catechin such as (−)-epicatechin-3-gallate and (−)-epigallo-catechin-3-gallate (EGCG); valoneic acid dilactone and gallagyldilactone isolated from the heartwood of Shorea laeviforia; Angelica koreana; Garden Balsam or Rose Balsam (Impatiens balsamina); pollen of turnip, turnip rape, fast plants, field mustard, or turnip mustard; dodder (Cuscuta reflexa); Euphorbia jolkinii; Lingzhi mushroom or Reishi mushroom (Ganoderma lucidum); ganoderic acid; Chinese Knotweed (Polygonum multiflorum); Black Pepper leaf extract (Piper nigrum); Red Stinkwood (Pygeum africanum); Saw Palmetto (Serenoa repens); a pine (Pinus sp. resin, active substance abietic acid); Ku Shen or Bitter root (Sophora flavescens); Japanese hedge parsley (Torilis japonica); Eastern Arborvitae or Northern Whitecedar (Thuja occidentalis); and Spore of Japanese climbing fern (Lygodium japonicum).

In some instances, the subject can be administered the additional pharmaceutical substance prior to the administration of the GnRH antagonist. In some instances, the subject can be administered the additional pharmaceutical substance after the administration of the GnRH antagonist.

In some exemplary embodiments, the subject can be administered testosterone prior to administration of the GnRH antagonist. In some exemplary embodiments, the subject has not been administered testosterone prior to administration of the GnRH antagonist.

In some instances, the GnRH antagonist can be administered at regular time intervals for an indefinite amount of time for the treatment of a chronic condition or disease. In some instances, the GnRH antagonist can be administered intermittently as needed.

### V. Indications

Disclosed herein are methods of administering a GnRH antagonist or a salt thereof to a subject. In some instances, the method is a method of treating, preventing, or at least partially ameliorating a disease or condition, or at least one symptom thereof. In some instances, the disease or condition can be benign prostatic hyperplasia; acute urinary retention; endometriosis; a cancer such as prostate, breast, or cervical cancer; a hormone imbalance; an androgen-sensitive condition; an estrogen sensitive condition; or a combination thereof. In some instances, the disease or condition may not be benign prostatic hyperplasia; acute urinary retention; endometriosis; a cancer such as prostate, breast, or cervical cancer; a hormone imbalance; an androgen-sensitive condition; an estrogen sensitive condition; or a combination thereof.

Administration of a first and second GnRH antagonist via a first and second route of entry, respectively, can be used to achieve an in vivo biological effect. In some instances, the effect can be a suppression of a hormone. In some cases, a hormone can be testosterone or dihydrotestosterone (DHT). In some instances, a hormone can be an estrogen, an estradiol, or progesterone.

In some instances, the administration can be used to reduce a concentration of testosterone or DHT to a concentration that is less than a concentration prior to the administration. Such a method can be used to at least partially ameliorate a disease or condition that can result from in an elevated level of testosterone or DHT. In some cases, the disease or condition can be hyperthyroidism, Grave's disease, precocious puberty, or a cancer such as prostate cancer. In some instances, the method of reducing a concentration of testosterone or DHT can be used as a method of chemical castration. In some instances, the method of reducing a concentration of testosterone or DHT can be used as a method of birth control.

In embodiments, administration of the GnRH antagonist can produce and/or maintain a blood plasma testosterone or DHT concentration of less than about 10 ng/mL, 9.5 ng/mL, 9 ng/mL, 8.5 ng/mL, 8 ng/mL, 7.5 ng/mL, 7 ng/mL, 6.5 ng/mL, 6 ng/mL, 5.5 ng/mL, 5 ng/mL, 4.5 ng/mL, 4 ng/mL, 3.5 ng/mL, 3 ng/mL, 2.5 ng/mL, 2 ng/mL, 1.5 ng/mL, 1 ng/mL, or 0.5 ng/mL. In embodiments, administration of the GnRH antagonist can produce or maintain a blood plasma testosterone or DHT concentration of about 10 ng/mL, 9.5 ng/mL, 9 ng/mL, 8.5 ng/mL, 8 ng/mL, 7.5 ng/mL, 7 ng/mL, 6.5 ng/mL, 6 ng/mL, 5.5 ng/mL, 5 ng/mL, 4.5 ng/mL, 4 ng/mL, 3.5 ng/mL, 3 ng/mL, 2.5 ng/mL, 2 ng/mL, 1.5 ng/mL, 1 ng/mL, or 0.5 ng/mL. In embodiments, administration of the GnRH antagonist can produce or maintain a blood plasma testosterone or DHT concentration of from about 0.5 ng/mL to about 10 ng/mL, from about 1 ng/mL to about 10 ng/mL, from about 1.5 ng/mL to about 10 ng/mL, from about 2 ng/mL to about 10 ng/mL, from about 2.5 ng/mL to about 10 ng/mL, from about 3 ng/mL to about 10 ng/mL, from about 3.5 ng/mL to about 10 ng/mL, from about 4 ng/mL to about 10 ng/mL, from about 4.5 ng/mL to about 10 ng/mL, from about 5 ng/mL to about 10 ng/mL, from about 5.5 ng/mL to about 10 ng/mL, from about 6 ng/mL to about 10 ng/mL, from about 6.5 ng/mL to about 10 ng/mL, from about 7 ng/mL to about 10 ng/mL, from about 7.5 ng/mL to about 10 ng/mL, from about 8 ng/mL to about 10 ng/mL, from about 8.5 ng/mL to about 10 ng/mL, from about 9 ng/mL to about 10 ng/mL, or from about 9.5 ng/mL to about 10 ng/mL.

In some exemplary embodiments, administration of the GnRH antagonist can maintain a blood plasma testosterone or DHT concentration of less than 0.5 ng/mL for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours after administration.

In some specific embodiments, the method can be used to at least partially ameliorate prostate cancer. In some cases, the method can include administration of a GnRH antagonist as a part of an androgen deprivation therapy. Administration of the GnRH antagonist can decrease levels of androgens such as testosterone and DHT as described above. Because androgens can stimulate prostate cancer cells to grow, suppression of androgens can cause an amelioration of prostate cancer by at least partially slowing a rate of growth of the prostate cancer cells, or producing a reduction in a size of a prostate. In some instances, the reduction of size can be at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some instances, the reduction of size can be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some instances, the reduction in size can occur after about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some instances, the reduction in size can occur after about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks.

In some instances, a prostate size can be measured using an ultrasound image. In some instances, a prostate size can be measured using a magnetic resonance imaging (MRI). In some instances, a prostate size can be determined manually by means of a rectal examination. In some instances, a prostate size can be determined digitally by means of a rectal examination. In some instances, a prostate size can be determined surgically.

In some instances, administration of a GnRH antagonist can reduce a size of a prostate after administration to a subject. In some cases, the GnRH agonist can be administered along with a neoadjuvant such as an anthracycline, a taxane, 5-fluorouracil, cyclophosphamide, carboplatin, or a combination thereof. In some cases, the GnRH can be the neoadjuvant.

In some instances, administration of a GnRH antagonist can be used to treat a disease or condition resulting from an enlarged prostate other than cancer. In some instances, the disease or condition can be benign prostatic hyperplasia, acute urinary retention, or a combination thereof.

Benign prostatic hyperplasia (BPH, otherwise known as an enlarged prostate) can occur in men as they age. Symptoms of BPH can include a weak or slow urinary system, a feeling of incomplete bladder emptying, difficulty urinating, frequent urination, and/or “dribbling” of urine. A GnRH antagonist can be used to at least partially slow a rate of growth of a prostate gland, thereby ameliorating the symptoms of BPH. In some cases, the reduction in size can be a reduction in mass. In some cases, the reduction in size can be a reduction in volume. In some instances, the reduction of size can be at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some instances, the reduction of size can be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some instances, the reduction in size can occur after about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some instances, the reduction in size can occur after about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks.

Acute urinary retention (AUR) is a condition characterized by an inability to voluntarily pass urine. Like BPH, AUR can be a result of an increase in size of a prostate gland. In some instances, AUR can occur in conjunction with BPH or prostate cancer. A GnRH antagonist can be used to at least partially slow a rate of growth of a prostate gland, thereby ameliorating the symptoms of AUR and increasing a rate of urine flow. In some instances, the increase in urine flow can be at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some instances, the increase in urine flow can be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%. In some instances, the increase in urine flow can occur after about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some instances, the increase in urine flow can occur after about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks.

In some cases, a rate or amount of urine flow can be determined using a urine flow test or assay. In some cases, the urine test can employ an electronic uroflowmeter. In some cases, the urine flow test is a urodynamic test. In some instances, the urodynamic test can comprise a measurement of urine flow rate and/or urine volume.

In some instances, treatment via administration of a GnRH antagonist described herein can decrease an amount of prostate specific antigen (PSA) in a subject. PSA is a substance made by cells in the prostate gland (both normal cells and cancer cells). PSA is mostly found in semen, but a small amount is also found in the blood. Incidence of an enlargement of a prostate, such as through a condition such as BPH or prostate cancer, can increase an amount of PSA in a subject. Therefore, PSA levels can be used as a metric for treatment of a condition resulting in the increase in PSA levels.

In embodiments, administration of the GnRH antagonist can reduce a concentration of PSA to less than about 10 ng/mL, 9.5 ng/mL, 9 ng/mL, 8.5 ng/mL, 8 ng/mL, 7.5 ng/mL, 7 ng/mL, 6.5 ng/mL, 6 ng/mL, 5.5 ng/mL, 5 ng/mL, 4.5 ng/mL, 4 ng/mL, 3.5 ng/mL, 3 ng/mL, 2.5 ng/mL, 2 ng/mL, 1.5 ng/mL, 1 ng/mL, or 0.5 ng/mL. In embodiments, administration of the GnRH antagonist can reduce a concentration of PSA to about 10 ng/mL, 9.5 ng/mL, 9 ng/mL, 8.5 ng/mL, 8 ng/mL, 7.5 ng/mL, 7 ng/mL, 6.5 ng/mL, 6 ng/mL, 5.5 ng/mL, 5 ng/mL, 4.5 ng/mL, 4 ng/mL, 3.5 ng/mL, 3 ng/mL, 2.5 ng/mL, 2 ng/mL, 1.5 ng/mL, 1 ng/mL, or 0.5 ng/mL. In embodiments, administration of the GnRH antagonist can reduce a concentration of PSA to an amount from about 0.5 ng/mL to about 10 ng/mL, from about 1 ng/mL to about 10 ng/mL, from about 1.5 ng/mL to about 10 ng/mL, from about 2 ng/mL to about 10 ng/mL, from about 2.5 ng/mL to about 10 ng/mL, from about 3 ng/mL to about 10 ng/mL, from about 3.5 ng/mL to about 10 ng/mL, from about 4 ng/mL to about 10 ng/mL, from about 4.5 ng/mL to about 10 ng/mL, from about 5 ng/mL to about 10 ng/mL, from about 5.5 ng/mL to about 10 ng/mL, from about 6 ng/mL to about 10 ng/mL, from about 6.5 ng/mL to about 10 ng/mL, from about 7 ng/mL to about 10 ng/mL, from about 7.5 ng/mL to about 10 ng/mL, from about 8 ng/mL to about 10 ng/mL, from about 8.5 ng/mL to about 10 ng/mL, from about 9 ng/mL to about 10 ng/mL, or from about 9.5 ng/mL to about 10 ng/mL.

In some specific embodiments, Teverelix TFA can provide a therapeutic benefit to achieve prolonged suppression of testosterone. Such a method can be employed to as a part of an androgen deprivation therapy, which is the commonly accepted treatment of patients with hormone sensitive prostate cancer. Based on preliminary analysis of the PK data and data collected in the Phase I study using this dosing regimen, it was expected that the increased loading dose of Teverelix LA used in this study (3 injections of 90 mg 24 h apart) would increase and prolong the initial peak and consequently increase the number of subjects castrated and increase the duration of the castration period.

While the three injections of 90 mg s.c. does increase the number of subjects castrated data suggested it would be desirable to increase the release rate in the early period (loading dose) after administration of teverelix TFA so that castration would occur almost immediately, specifically within 48 to 72 hours without the flair seen with GnRH agonists and be maintained throughout the dosing interval this is especially true for the first dosing interval. While the three consecutive 90 mg doses may adequately address the therapeutic requirements it may be too cumbersome in a clinical setting. However, dual administration of teverelix through a combination of intramuscular and subcutaneous injection can have the surprising and unexpected result of providing chemical castration for a prolonged period following the initial dual administration.

In some instances, the duration of chemical castration can be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some instances, the duration of chemical castration can be at least about 1, 2, 3, 4, 5, 6, 7, 8, 9, 10, 11, or 12 weeks.

In some exemplary embodiments, the method is a method of chemical castration comprising:
(a) an intramuscular administering of teverelix trifluoroacetate to a subject; and
(b) a subcutaneous administering of teverelix trifluoroacetate to the subject,
   where the first administering and the second administering are performed on the same day, and where a blood plasma testosterone concentration in the subject of less than about 0.5 ng/mL is achieved by about 48 hours after the intramuscular administering and the subcutaneous administering.

In some exemplary embodiments, the method is a method of treating benign prostatic hyperplasia, comprising:
(a) an intramuscular administering of teverelix trifluoroacetate to a subject; and
(b) a subcutaneous administering of teverelix trifluoroacetate to the subject,
   where the first administering and the second administering are performed on the same day, and where an increase in urine flow in the subject of at least about 10% is achieved by about two weeks after the intramuscular administering and the subcutaneous administering.

In some instances, the method is a method of treating a disease or condition associated with an increase in blood plasma levels of an estrogen in a female subject. In some cases, administration of a GnRH antagonist can decrease the level of estrogen in the subject, thereby at least partially ameliorating the disease or condition, or a symptom thereof. In some cases, the disease or condition is a cancer such as breast cancer. In some cases, the method can be a method of reducing pain in a subject due to a condition such as endometriosis. In some instances, the estrogen can be an estradiol such as 17α-estradiol, 17β-estradiol, or ethinylestradiol.

In embodiments, administration of the GnRH antagonist can produce and/or maintain a blood plasma concentration of an estrogen of less than about 10 ng/mL, 9.5 ng/mL, 9 ng/mL, 8.5 ng/mL, 8 ng/mL, 7.5 ng/mL, 7 ng/mL, 6.5 ng/mL, 6 ng/mL, 5.5 ng/mL, 5 ng/mL, 4.5 ng/mL, 4 ng/mL, 3.5 ng/mL, 3 ng/mL, 2.5 ng/mL, 2 ng/mL, 1.5 ng/mL, 1 ng/mL, or 0.5 ng/mL. In embodiments, administration of the GnRH antagonist can produce or maintain a blood plasma concentration of an estrogen of about 10 ng/mL, 9.5 ng/mL, 9 ng/mL, 8.5 ng/mL, 8 ng/mL, 7.5 ng/mL, 7 ng/mL, 6.5 ng/mL, 6 ng/mL, 5.5 ng/mL, 5 ng/mL, 4.5 ng/mL, 4 ng/mL, 3.5 ng/mL, 3 ng/mL, 2.5 ng/mL, 2 ng/mL, 1.5 ng/mL, 1 ng/mL, or 0.5 ng/mL. In embodiments, administration of the GnRH antagonist can produce or maintain a blood plasma concentration of an estrogen of from about 0.5 ng/mL to about 10 ng/mL, from about 1 ng/mL to about 10 ng/mL, from about 1.5 ng/mL to about 10 ng/mL, from about 2 ng/mL to about 10 ng/mL, from about 2.5 ng/mL to about 10 ng/mL, from about 3 ng/mL to about 10 ng/mL, from about 3.5 ng/mL to about 10 ng/mL, from about 4 ng/mL to about 10 ng/mL, from about 4.5 ng/mL to about 10 ng/mL, from about 5 ng/mL to about 10 ng/mL, from about 5.5 ng/mL to about 10 ng/mL, from about 6 ng/mL to about 10 ng/mL, from about 6.5 ng/mL to about 10 ng/mL, from about 7 ng/mL to about 10 ng/mL, from about 7.5 ng/mL to about 10 ng/mL, from about 8 ng/mL to about 10 ng/mL, from about 8.5 ng/mL to about 10 ng/mL, from about 9 ng/mL to about 10 ng/mL, or from about 9.5 ng/mL to about 10 ng/mL.

In some exemplary embodiments, administration of the GnRH antagonist can maintain a blood plasma concentration of an estrogen of less than 0.5 ng/mL for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours after administration.

### VI. Kits

Kits can comprise at least one GnRH antagonist described herein or a salt thereof, and instructions for use thereof. In some aspects, the GnRH antagonist or salt thereof can be packaged in a container.

In some aspects, the instructions for use can provide instructions for treating a disease or condition, or a symptom thereof. In some instances, the disease or condition can comprise benign prostatic hyperplasia; acute urinary retention; endometriosis; a cancer such as prostate or breast cancer; or any combination thereof.

In some aspects, a kit can comprise at least one syringe and needle for administration of a GnRH antagonist. In some exemplary embodiments, a pair of needles can be provided of proper length for injection via intramuscular and subcutaneous injection. In some instances, a single needle can be used to administer a GnRH antagonist subcutaneously and intramuscularly. Instructions for such an administration can be packaged in the kit.

In some instances, a GnRH antagonist can be provided as a lyophilized sample contained in a vial. A kit can comprise a vial of sterile water for reconstitution/resuspension of the lyophilized GnRH antagonist, as well as instructions for preparation. In other instances, a syringe comprising an integrated water chamber can be provided to resuspend the lyophilized GnRH antagonist within the syringe prior to administration.

Methods of making the kit can include placing at least one GnRH antagonist described herein or a salt thereof in a container for packaging. The method can further comprise the inclusion of instructions for use. In some cases, the instructions for use can provide instructions for treating a disease or condition, or a symptom thereof.

### VII. Examples

### Example 1—Teverelix SC Injection

In a Phase I study (D-84812-Z012), doses of up to 90 mg of Teverelix TFA (trifluoroacetate) that were administered to 32 healthy male volunteers as a single s.c. injection were tolerated well, with no critical findings regarding laboratory and clinical safety. The pharmacodynamics of teverelix showed a rapid marked suppression of LH, testosterone and, to a lesser extent, FSH. Duration of at least half-maximum testosterone suppression increased with dose. The pharmacokinetics of teverelix showed an initial increase followed by a slow and controlled release of drug. The half-life ranged between 252 h and 331 h. The maximum plasma concentrations were observed 1.75 h to 3 h after s.c. injection. Dose proportionality was demonstrated between the 60 mg and 90 mg doses of Teverelix TFA. Only the low doses of 10 mg and 30 mg Teverelix LA appeared to be insufficient to attain hormone suppression over a long period.

In a second Phase I study (EP-24332T-A004), a second injection was administered 24 h or 48 h after the first in order to increase and prolong the initial peak, as well as increase the teverelix level and consequently the number of subjects castrated. As the previous Phase I study had shown 10 mg and 30 mg to be insufficient to provide a long-term suppression, 2 doses of 60 mg or 90 mg (6 subjects per group) or placebo (4 subjects) were administered at a 24 h interval and 2 doses of 90 mg at a 48 h interval. Teverelix was tolerated well and only mild local indurations at the injection sites were observed. No critical safety findings were reported. The pharmacodynamics of teverelix showed that the mean levels of testosterone were maintained at 40% and 60% of the baseline value for more than 4 weeks in the 60 mg/60 mg and 90 mg/90 mg groups, respectively. The protocol of the study included 6 subjects who received 90 mg Teverelix TFA on D1; D2; D3. In this group, suppression of testosterone to below castration level (<0.5 ng/mL) was achieved in 5 out of 6 subjects. However, mean testosterone level was above castration level as the pharmacodynamic response in one subject was different from the pharmacodynamic response in the remaining 5 subjects.

### Example 2—Teverelix IM Injection (2×90 mg, Days 1 and 8)

In order to test the hypothesis that and IM injection of a GnRH antagonist could be useful as part of a prostate cancer regimen, a Phase II Multicenter, Open-label Study Investigating the Pharmacokinetics, Pharmacodynamics, Efficacy and Safety of a Teverelix 90 mg, TFA formulation was conducted.

The primary objective was to assess the duration of action of an initial “loading” dose regimen of Teverelix TFA delivered IM in terms of suppression of testosterone to below castrate level (0.5 ng/mL [2 nmol/L]). The secondary objectives were to assess the pharmacodynamics of teverelix in terms of ability to suppress and to maintain plasma testosterone levels below castration level (<0.5 ng/mL [2 nmol/L]) for 3 weeks; to assess the effects on luteinising hormone (LH) and prostate specific antigen (PSA); and to assess the safety of Teverelix in terms of incidence of local and systemic tolerability (adverse events [AEs] and changes in laboratory parameters).

This was a multicenter, open-label, non-controlled study in 14 patients diagnosed with advanced prostate cancer. Patients were screened for inclusion in the study between 1 and 7 days before first administration. Each patient received 2 intramuscular (i.m.) injections of 90 mg Teverelix TFA, 7 days apart, on Study Days 1 and 8. Patients attended the hospital unit on Study Days 1, 2, 3, 5, 8, 9, 10, 12 and 14 and then weekly until, after Week 3, two consecutive, increasing determinations of testosterone concentration above castration level were recorded, with the second determination of testosterone above 2 ng/mL. Blood samples were taken for the evaluation of teverelix, testosterone and LH in plasma, and PSA levels. Safety and tolerability were assessed throughout the study by monitoring local and systemic tolerability (AEs, clinical laboratory tests, vital signs an electrocardiogram [ECG]).

Pharmacokinetics of Teverelix

The geometric means for Cmax(obs), AUC(0-t), and AUC(0-∞) (along with respective coefficients of variations presented in parenthesis); Tmax median (along with the range of Tmax values presented in parenthesis); and arithmetic mean for T_{1/2,elim} (along with standard deviation presented in parenthesis) are presented in Table 1 for each dose of teverelix IM.

TABLE 1
Plasma PK Parameter Estimates for Teverelix TFA for Day 1 and Day 8

| | | Cmax (obs) | AUC (0-t) | AUC (0-∞) | | |
|---|---|---|---|---|---|---|
| | | (ng/mL) | (ng · h/mL) | (ng · h/mL) | Tmax (obs) | |
| | | Geometric | Geometric | Geometric | (h) | T_{1/2,elim} (h) |
| | | mean | mean | mean | Median | Arithmetic |
| Treatment | N | (CV %) | (CV %) | (CV %) | (range) | mean (SD) |
| Day 1(Dose 1): 1 × 90 mg | 14 | 21.5 | 1734 | 4610* | 2.00 | 166.60* |
| Teverelix TFA (i.m.) | | (69.5) | (101.1) | (86.1) | (1.00-24.00) | (72.9) |
| Day 8 (Dose 2): 1 × 90 mg | 14 | 38.0 | 8355 | 9377 | 1.00 | 404.43 |
| Teverelix TFA (i.m.) | | (33.8) | (22.2) | (20.0) | (1.00-24.00) | (113.15) |

Note:
*Indicates n = 8.

The combined geometric means for AUC and AUC(∞) (along with respective coefficients of variations presented in parenthesis); and arithmetic mean for apparent clearance (CL/F) (along with standard deviation presented in parenthesis) for the two i.m. doses of teverelix are presented in Table 2.

TABLE 2
Plasma PK Parameter Estimates for Teverelix

| | | AUC | AUC(∞) | CL/F |
|---|---|---|---|---|
| | | (ng · h/mL) | (ng · h/mL) | (mL/h) |
| | | Geometric | Geometric | Arithmetic |
| | | mean | mean | mean |
| Treatment | N | (CV %) | (CV %) | (SD) |
| 2 × 90 mg | 14 | 10480 | 11510 | 14940 |
| Teverelix TFA (i.m.) | | (27.3) | (24.6) | (3720) |

Pharmacokinetic Conclusions

Secondary teverelix concentration maxima were apparent in concentration vs time profiles more than 24 h after the administration of the final dose, indicating the sustained release of drug from the long-acting teverelix formulation.

Maximum concentrations for the second dose were greater than the corresponding concentrations following the first dose (Cmax(obs)) as a result of super-imposition of quantifiable teverelix from the second dose onto the preceding concentration-time profile. This is consistent with the quantifiable predose teverelix samples observed prior to administration of the second dose.

Estimates of T_{1/2,elim} for teverelix were longer for the second dose compared with the first dose. The blood sampling period after the second dose was longer than that after the first dose as samples were taken until the criteria for the determination of the PD parameter T_{esc} were achieved. Therefore, the longer T_{1/2,elim} estimates observed for the second dose probably reflects the later time ranges used to determine the lambda-z slope for plasma profiles.

Determination of Testosterone Levels

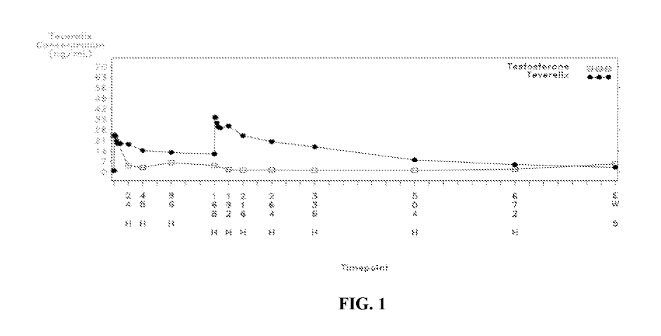

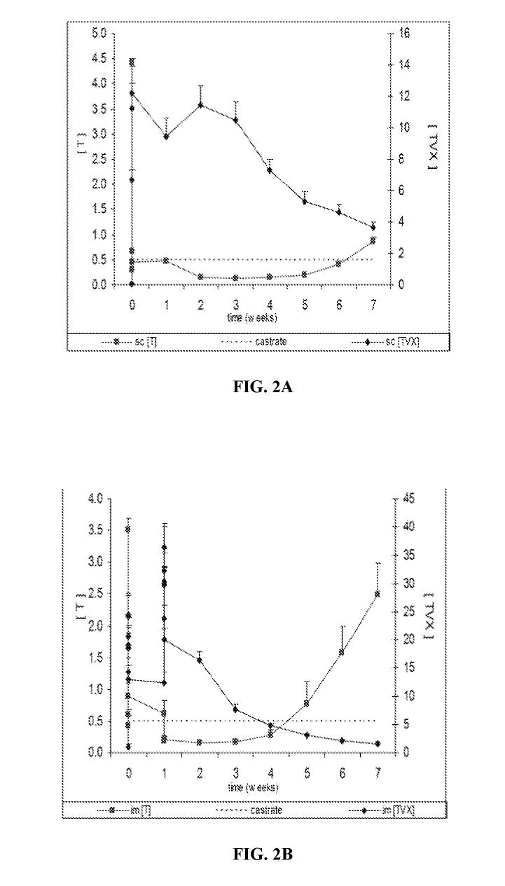

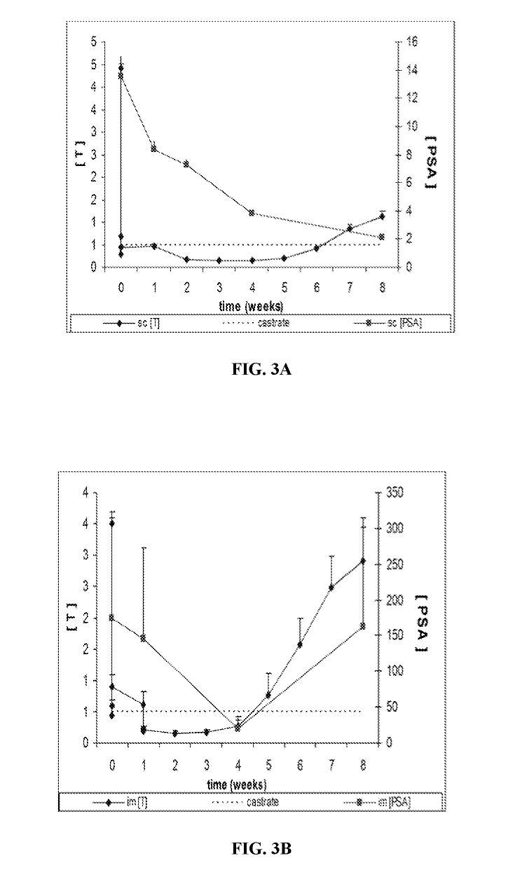

Mean concentrations of testosterone decreased rapidly after administration of teverelix (Day 2 mean [SD] change from baseline was −2.906 [0.545] ng/mL, a decrease from baseline of approximately 83%). Suppression of serum testosterone concentrations continued until at least the end of Week 5 (see FIG. 1) and individual serum concentrations for all patients, except for Patient 01, remained below 2 ng/mL. Patient 01 had serum testosterone concentrations of 0.27, 1.25 and 4.97 ng/mL at the end of Weeks 3, 4 and 5, respectively.

Determination of Teverelix (ng/mL) and Testosterone (ng/mL), Mean Values on a Linear Scale

By Day 9 (i.e. 2 days after administration of the second dose) all patients' testosterone concentration had decreased to below a threshold considered to be biochemical castration (<0.5 ng/mL); see FIG. 1.

The time of onset of castration (T_{cast}), time of escape from castration (T_{esc}), and duration of castration (DC) are summarized in Table 3.

TABLE 3
Pharmacodynamic Parameters Tcast, Tesc and DC: ITT and PP Datasets

| | | T_{cast} | T_{esc} | DC |
|---|---|---|---|---|
| Treatment | Statistic | (Days) | (Days) | (Days) |
| 2 × 90 mg | Mean (SD) | 2.40 (2.52) | 46.17 (10.88) | 34.77 (9.28) |
| Teverelix TFA | Median (range) | 1.30 (0.9-8.0) | 46.20 (29.4-75.2) | 34.90 (20.8-57.7) |

The mean (SD) time taken to achieve castration (T_{cast}) was 2.40 (2.52) days. T_{cast} ranged from 0.9 to 8 days and was within 2 days of administration of the first dose of teverelix for 11/14 patients. T_{cast} was longer than 2 days for 3 patients. T_{cast} for Patients 03, 06 and 09 was estimated as 8.0, 6.0 and 6.9 days, respectively.

The mean (SD) duration of castration (DC) was 34.77 (9.28) days. DC ranged from 20.8 to 57.7 days and was longer than 4 weeks for 11/14 patients. DC was estimated to be less than 4 weeks for 3 patients: DC for Patients 01, 03 and 08 was 21.3, 20.8 and 26.0 days, respectively. The mean (SD) time to escape from castration (T_{esc}) was 46.17 (10.88) days and ranged from 29.4 to 75.2 days after the first administration of Teverelix. Escape from castration followed a steady decrease in plasma teverelix concentration.

Eight out of the 14 patients (57.1%) experienced transient breakthrough episodes during castration where concentrations of testosterone increased to above 0.5 ng/mL (Patients 02, 03, 05, 06, 07, 12, 13 and 14). In all instances, testosterone concentrations remained below 2 ng/mL and did not proceed to meet the criteria for escape from castration. For 6/8 patients, breakthrough episodes occurred before administration of the second dose of Teverelix LA, and all episodes occurred before the end of Week 2. Breakthrough episodes varied in duration from between 2.3 and 5.4 days (mean [SD] duration=3.60 [1.28] days).

Comparison of s.c. injection 90 mg days 1, 2 and 3 vs. IM injection day 1 and 8.

TABLE 4
Summary of Clinical Studies

| Parameter | A013 | A014 |
|---|---|---|
| Onset of | 1.77 days | 2.40 days |
| castration | | |
| Mean duration of | 55.32 days | 34.77 days |
| castration | (range 4-14 weeks) | (range 3-8 weeks) |
| Mean escape | 70.14 days | 46.17 days |
| from castration | | |
| T below baseline | ≥ End Week 6 | ≥ End Week 4 |
| LH | Decreased following first | Decreased appreciably |
| | administration of | following administration |
| PSA | teverelix on Day 1 and | of Teverelix LA and |
| | remained below baseline | remained below |
| | until at least the end of | baseline values untli at |
| | Week 6 | least the end of Week 5 |
| | | (LH) and Week 4 (PSA) |

| | |
|---|---|
| Tolerability | Very good/good for all patients |

From the above it is clear that both the 3×90 mg s.c. dosing regimen and the 2×90 mg IM dosing regimen would meet the primary objective of castrating the prostate cancer patients within 48 to 72 hours and maintain castration for the full 4 week dosing interval. However, it would not meet the practical criterion of being able to treat the patient with a single visit once every 4 weeks.

### Example 3—GnRH IM/SC Combination (2×90 mg, Day 1)

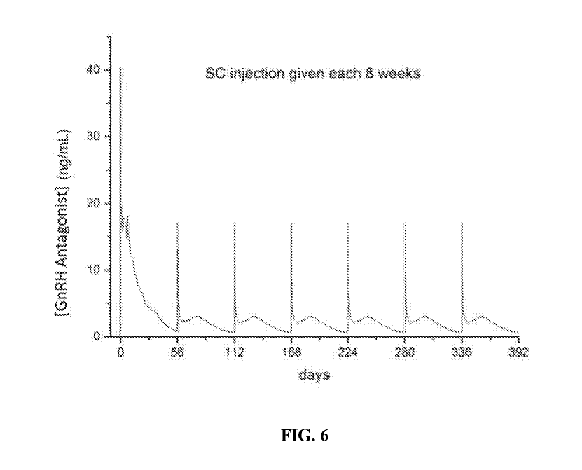

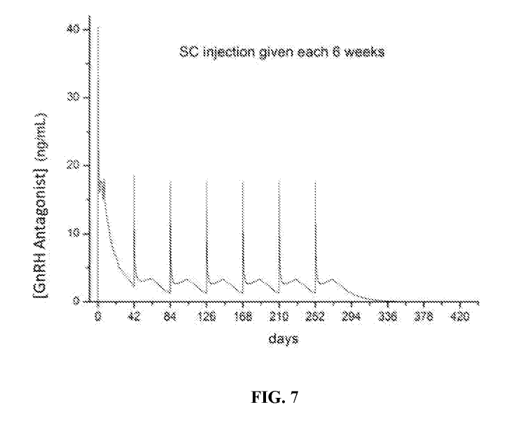

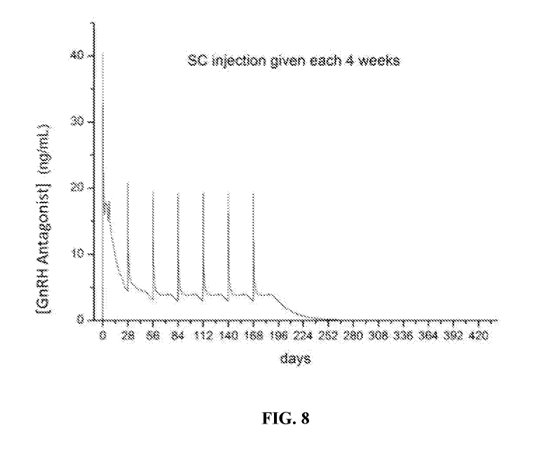

Mean data from the i.m. and s.c. injection studies was used to predict the PK profile for a combined i.m. and s.c. dosing regimen of an administration of a GnRH antagonist. A PK profile for three potential dosing regimens was constructed: Scenario A (90 mg IM+90 mg SC, then 90 mg SC every 56 days; FIG. 6); Scenario B (90 mg IM+90 mg SC, then 90 mg SC every 42 days; FIG. 7); and Scenario C (90 mg IM+90 mg SC, then 90 mg SC every 28 days; FIG. 8). The steady state Cmax, Cmin, AUC and Cavg are summarized below:

TABLE 5
Simulated PK/PD parameters for s.c. and i.m. dual injection

| | C_{max,ss} | C_{min,ss} | AUCₛₛ | C_{avg,ss} |
|---|---|---|---|---|
| | (ng/mL) | (ng/mL) | (ng × days/mL) | (ng/mL) |
| Scenario A | 16.95 (15-20) | 0.56 (0.3-0.7) | 115.57 (110-120) | 2.06 (1.5-3.5) |
| Scenario B | 17.66 (15-20) | 1.27 (0.8-1.7) | 115.57 (110-120) | 2.75 (2.2-3.2) |
| Scenario C | 19.33 (15-20) | 2.95 (2.5-3.5) | 115.57 (110-120) | 4.13 (3.6-4.6) |

The combined administration of the IM and SC injection, serves to provide efficient serum concentration already during the first dosing interval. As expected, shortening of the steady state dosing interval can linearly increase the average serum concentrations.

### Example 4—Teverelix IM/SC Combination (2×90 mg, Day 1)

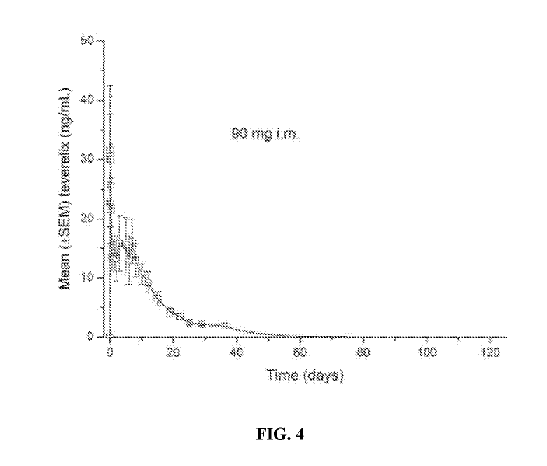

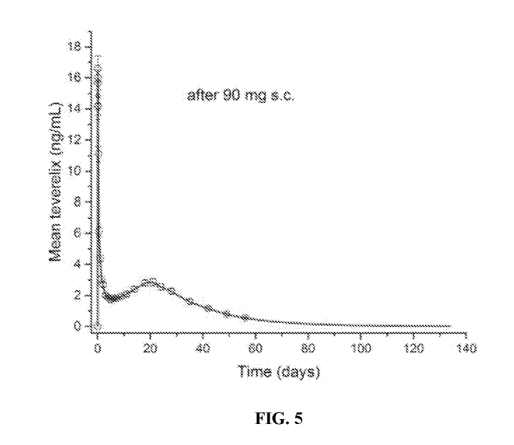

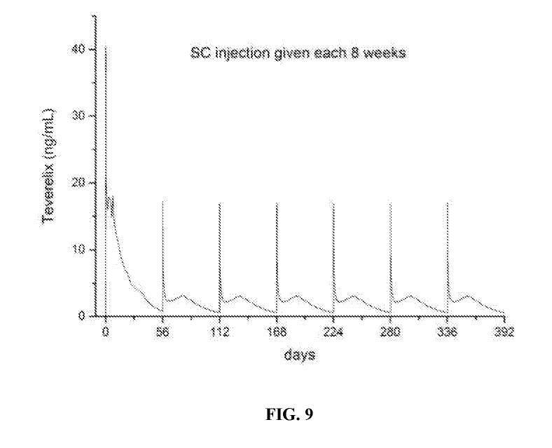

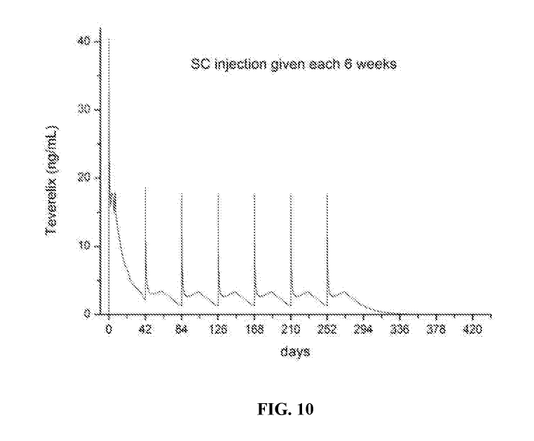

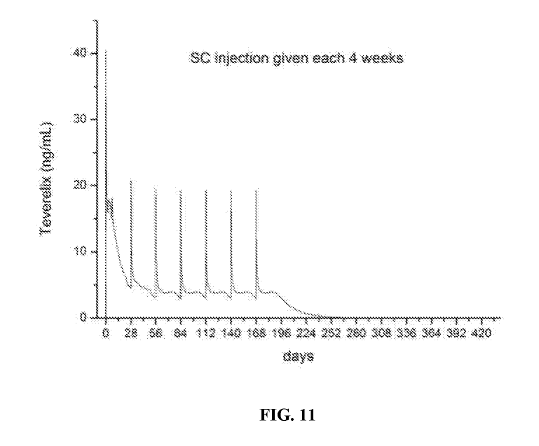

Mean data from the i.m. and s.c. injection studies was used to predict the PK profile for a combined i.m. and s.c. dosing regimen. FIGS. 4 and 5 depict the mean serum concentrations of teverlix (ng/mL) after a single i.m. and s.c. injection, respectively. This data was used to construct a PK profile for three potential dosing regimens: Scenario A (90 mg IM+90 mg SC, then 90 mg SC every 56 days; FIG. 9); Scenario B (90 mg IM+90 mg SC, then 90 mg SC every 42 days; FIG. 10); and Scenario C (90 mg IM+90 mg SC, then 90 mg SC every 28 days; FIG. 11). The steady state Cmax, Cmin, AUC and Cavg are summarized below:

TABLE 6
Simulated PK/PD parameters for s.c. and i.m. dual injection of Teverelix TFA

| | Cmax, ss | Cmin, ss | AUCss | Cavg, ss |
|---|---|---|---|---|
| | (ng/mL) | (ng/mL) | (ng × days/mL) | (ng/mL) |
| Scenario A | 16.95 (15-20) | 0.56 (0.3-0.7) | 115.57 (110-120) | 2.06 (1.5-3.5) |
| Scenario B | 17.66 (15-20) | 1.27 (0.8-1.7) | 115.57 (110-120) | 2.75 (2.2-3.2) |
| Scenario C | 19.33 (15-20) | 2.95 (2.5-3.5) | 115.57 (110-120) | 4.13 (3.6-4.6) |

The combined administration of the IM and SC injection of teverelix TFA serves to provide efficient serum concentration already during the first dosing interval. As expected, shortening of the steady state dosing interval can linearly increase the average serum concentrations

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

1.-19. (canceled)

20. A method comprising:
(a) administering intramuscularly a therapeutically effective amount of a first GnRH antagonist salt to a subject, and
(b) administering subcutaneously a therapeutically effective amount of a second GnRH antagonist or a salt thereof to the subject,
wherein the administering of the first GnRH antagonist salt and the administering of the second GnRH antagonist salt are performed within about 168 hours of one another.

21. The method of claim 20, wherein the administering of the first GnRH antagonist salt and the administering of the second GnRH antagonist salt are performed within 24 hours of one another.

22.-24. (canceled)

25. The method of claim 20, wherein the administering intramuscularly and the administering subcutaneously independently comprise an injection.

26. (canceled)

27. (canceled)

28. The method of claim 20, wherein the method is a method of at least partially ameliorating a disease.

29.-39. (canceled)

40. The method of claim 20, wherein the subject is human.

41. (canceled)

42. The method of claim 20, wherein the subject is in need thereof.

43.-46. (canceled)

47. The method of claim 20, wherein the administering intramuscularly and the administering subcutaneously provide a blood plasma testosterone concentration less than about: 0.5 ng/mL as measured by a liquid chromatography/mass spectrometry (LC-MS) assay.

48. (canceled)

49. The method of claim 47, wherein a blood plasma testosterone concentration of less than about 0.5 ng/mL is achieved by about: 24 hours after the administering subcutaneously and the administering intramuscularly.

50.-64. (canceled)

65. The method of claim 20, wherein the first GnRH antagonist salt and the second GnRH antagonist salt are independently a peptide salt.

66. The method of claim 65, wherein the first GnRH antagonist salt and the second GnRH antagonist salt are independently in a crystalline form, wherein the crystalline form is in suspension in a liquid.

67. The method of claim 66, wherein the crystalline form is a microcrystalline form.

68. The method of claim 65, wherein the first GnRH antagonist salt and the second GnRH antagonist salt independently comprise a peptide salt comprising a counter-ion, wherein the counter-ion is the conjugate base of an acid having a pKa of less than about 2.

69. (canceled)

70. (canceled)

71. The method of claim 20, wherein the first GnRH antagonist salt and the second GnRH antagonist salt are independently a teverelix salt.

72. The method of claim 71, wherein the first GnRH antagonist salt and the second GnRH antagonist salt are independently teverelix trifluoroacetate.

73. The method of claim 72, wherein the administering intramuscularly and the administering subcutaneously independently comprise administering a dosage ranging from about 30 mg to about 240 mg of teverelix trifluoroacetate.

74. (canceled)

75. The method of claim 71, further comprising providing a blood plasma teverelix concentration in the subject of at least about 9 ng/mL.

76. The method of claim 71, further comprising providing a maximal plasma concentration (Cmax) of teverelix in the subject of at least about 19 ng/mL.

77. (canceled)

78. (canceled)

79. The method of claim 20, wherein the subject is suspected of having a disease.

80. (canceled)

81. The method of claim 20, further comprising administering an additional pharmaceutical substance.

82.-94. (canceled)

95. The method of claim 66, wherein the first GnRH antagonist salt and the second GnRH antagonist salt is in a formulation further comprising an isotonic agent.

96.-105. (canceled)
